(19) United States
(12) Patent Application Publication
(10) Pub. No.: US 2024/0369530 A1
(43) Pub. Date: Nov. 7, 2024

(54) MULTIPLEXED ELECTRONIC IMMUNOASSAY USING ENZYMATICALLY AMPLIFIED METALLIZATION ON MICROPARTICLES

(71) Applicant: Georgia Tech Research Corporation, Atlanta, GA (US)

(72) Inventors: Aniruddh Sarkar, Atlanta, GA (US); Madeline Hoyle, Atlanta, GA (US); Neda Rafat, Atlanta, GA (US); Josiah Rudge, Atlanta, GA (US)

(21) Appl. No.: 18/685,948

(22) PCT Filed: Sep. 23, 2022
(86) PCT No.: PCT/US2022/076922

§ 371 Date: Feb. 23, 2024

Related U.S. Application Data
(60) Provisional application No. 63/247,498, filed on Sep. 23, 2021.

(51) Int. Cl. G01N 33/487; G01N 33/543; G01N 33/552; G01N 33/553; G01N 33/58

(52) U.S. Cl. CPC G01N 33/48707; G01N 33/54346; G01N 33/552; G01N 33/553; G01N 33/588

(57) ABSTRACT

An exemplary embodiment of the present disclosure provides a composition comprising a bead having on its surface a molecule of interest configured to bind with a target molecule in a biological sample, a probe molecule configured to bind with the target molecule, and a metal particle configured to bind with the probe molecule. In any of the embodiments disclosed herein, the bead can comprise an identifying characteristic. In any of the embodiments disclosed herein, the metal particle can be configured to modify an electrical property of the bead. In any of the embodiments disclosed herein, the molecule of interest can comprise one or more of: an antigen, an epitope, an antibody, protein, DNA, RNA, virus, bacteria, and mammalian cell. In any of the embodiments disclosed herein, the target molecule can comprise one or more of: an antibody, a paratope, a protein, DNA, RNA, a virus, a bacterium, and a mammalian cell.

### CROSS-REFERENCE TO RELATED APPLICATIONS

This application claims the benefit of U.S. Provisional Application Ser. No. 63/247,498, filed on 23 Sep. 2021, which is incorporated herein by reference in its entirety as if fully set forth below.

### FEDERALLY SPONSORED RESEARCH STATEMENT

This invention was made with government support under grant/award number NIH 1R01A1152158-01 awarded by the National Institutes of Health. The government has certain rights in the invention.

### FIELD OF THE DISCLOSURE

The various embodiments of the present disclosure relate generally to medical diagnostics, and more particularly to immunoassay based diagnostic tests.

### BACKGROUND

Aspects of the disclosed technology improve bead-based immunoassays. Bead-based immunoassays are widely used in biological research and clinical diagnostics as they provide excellent sensitivity, speed, and extended dynamic range due to their enhanced binding kinetics and ease of automation. They also enable highly multiplexed assays via the simultaneous measurement of multiple markers from a single sample using barcoded beads. However current bead-based assays usually require expensive and bulky optical instrumentation (e.g., lasers/photomultiplier tubes, etc.).

These immunoassays can require the use of expensive fluorescent markers and optical elements for readout. Optical elements are not as tough or durable as electrical elements and can be more difficult to transport. The optical detection systems required, such as flow cytometers are complex, bulky and expensive as they rely on lasers, photodetectors, and beam shaping lenses. This hinders their use especially in the context of widespread infectious diseases such as COVID-19.

Cheap, durable, and widespread diagnostic methods are needed for to track the spread of infectious diseases.

### BRIEF SUMMARY

An exemplary embodiment of the present disclosure provides a composition comprising a bead having on its surface a molecule of interest configured to bind with a target molecule in a biological sample, a probe molecule configured to bind with the target molecule, and a metal particle configured to bind with the probe molecule.

In any of the embodiments disclosed herein, the bead can comprise an identifying characteristic. The identifying characteristic can be one or more of: an electrical impedance signature, optical barcoding, shape, size, porosity, conductivity, permittivity, permeability, and stiffness.

In any of the embodiments disclosed herein, the metal particle can be configured to modify an electrical property of the bead.

In any of the embodiments disclosed herein, the bead can comprise one or more of: carboxylated polystyrene, aminated polystyrene, glass, silica, polymethyl methacrylate, and hydrogels.

In any of the embodiments disclosed herein, the bead can be functionalized using EDC-NHS (N-ethyl-N-(3-(dimethylamino) propyl) carbodiimide/N-hydroxy succinimide) chemistry.

In any of the embodiments disclosed herein, the bead can have a spherical shape. The bead can have a diameter of approximately 3 μm.

In any of the embodiments disclosed herein, the bead can have a diameter of approximately 5 μm.

In any of the embodiments disclosed herein, the bead can have a diameter of approximately 10 μm.

In any of the embodiments disclosed herein, the bead can have a diameter of approximately 20 μm.

In any of the embodiments disclosed herein, the bead can have a diameter of from about 3 μm to about 20 μm.

In any of the embodiments disclosed herein, the molecule of interest can comprise one or more of: an antigen, an epitope, an antibody, protein, DNA, RNA, virus, bacteria, and mammalian cell.

In any of the embodiments disclosed herein, the target molecule can comprise one or more of: an antibody, a paratope, a protein, DNA, RNA, a virus, a bacterium, and mammalian cell.

In any of the embodiments disclosed herein, the biological sample can comprise one or more of: serum, whole blood, saliva, urine, and cerebrospinal fluid.

In any of the embodiments disclosed herein, the composition can further comprise a buffer. The buffer can be a phosphate buffer having a pH of from about 6.0 to about 7.0. The buffer can be a phosphate buffer comprising a pH of approximately 6.4.

In any of the embodiments disclosed herein, the probe molecule can comprise one or more of: horseradish peroxidase and alkaline phosphatase.

An exemplary embodiment of the present disclosure provides a method for measuring biomarkers, the method comprising selectively metalizing a bead, moving the metalized bead past an electrical measuring device, and measuring an electrical property of the metalized bead.

In any of the embodiments disclosed herein, selectively metalizing the bead can comprise allowing a molecule of interest disposed on a surface of the bead to bind with a target molecule in a biological sample, allowing a probe molecule to bind with the target molecule that is bound to the bead, and allowing a metal particle to bind with the probe molecule that is bound to the target molecule to modify the electrical property of the bead.

In any of the embodiments disclosed herein, allowing the molecule of interest to bind with the target molecule can comprise incubating the bead in a sample solution comprising the biological sample.

In any of the embodiments disclosed herein, incubating the bead in the sample solution can last for approximately 1800 seconds at approximately room temperature.

In any of the embodiments disclosed herein, allowing the probe molecule to bind with the target molecule can comprise incubating the bead bound to the target molecule in a probe solution comprising the probe molecule.

In any of the embodiments disclosed herein, incubating the bead bound to the target molecule in the probe solution can last for 3600 seconds at approximately room temperature.

In any of the embodiments disclosed herein, allowing the metal particle to bind with the probe molecule that is bound to the target molecule can comprise enzymatically depositing the metal particle on the probe molecule.

In any of the embodiments disclosed herein, the metal particle can comprise one or more of: a silver nanoparticle, a gold nanoparticle, and an iron nanoparticle

In any of the embodiments disclosed herein, measuring an electrical property of the metalized bead can comprise passing the metalized bead through a sensing zone disposed between an inlet and an outlet: and measuring an impedance across the sensing zone.

In any of the embodiments disclosed herein, the inlet can have a pyramidal shape. The outlet can have a pyramidal shape. The sensing zone can have a square shape.

In any of the embodiments disclosed herein, passing the bead through the sensing zone can comprise flowing a bead suspension through the sensing zone, the metalized bead suspended in the bead suspension.

In any of the embodiments disclosed herein, the bead suspension can be flowed at a rate of approximately 12 microliters per minute.

In any of the embodiments disclosed herein, measuring the impedance across the sensing zone can be performed with a lock-in amplifier.

In any of the embodiments disclosed herein, measuring the impedance can comprise sampling at approximately 100,000 samples per second at one or more frequencies. The one or more frequencies can be between approximately 45 kilohertz and 35 megahertz.

In any of the embodiments disclosed herein, the method can further comprise comparing the electrical property of the selectively metalized bead to an electrical property of a non-metalized bead.

In any of the embodiments disclosed herein, the method can comprise identifying a characteristic of the bead. The characteristic of the bead can be one or more of: an electrical impedance signature, optical barcoding, shape, size, porosity, conductivity, permittivity, permeability, and stiffness.

In any of the embodiments disclosed herein, the method can further comprise functionalizing the bead using EDC-NHS (N-ethyl-N′-(3-(dimethylamino) propyl) carbodiimide/N-hydroxy succinimide) chemistry.

An exemplary embodiment of the present disclosure provides a system for detecting biomarkers. The system can comprise a composition, an aperture, and an electrical property sensor. The composition can comprise a bead having on its surface a molecule of interest configured to bind with a target molecule in a biological sample, a probe molecule configured to bind with the target molecule, and a metal particle configured to bind with the probe molecule. The aperture can comprise an inlet, an outlet; and a sensing zone disposed between the inlet and the outlet, wherein the aperture is configured to pass the composition from the inlet, through the sensing zone, and to the outlet. The electrical property sensor can be disposed proximate the sensing zone and configured to sense an electrical property of the bead.

In any of the embodiments disclosed herein, the inlet can have a pyramidal shape. The outlet can have a pyramidal shape. The sensing zone can have a square shape.

In any of the embodiments disclosed herein, the sensing zone can be approximately 30 μm by 30 μm.

In any of the embodiments disclosed herein, the aperture can be configured to flow the composition at a rate of approximately 12 microliters per minute.

In any of the embodiments disclosed herein, the electrical property sensor can comprise a lock-in amplifier.

In any of the embodiments disclosed herein, the electrical property sensor further can comprise a processor configured to compare the electrical property of the bead to an electrical property of a non-metalized bead. The electrical property sensor can be configured to identify a characteristic of the bead, and the characteristic of the bead can be one or more of: an electrical impedance signature, optical barcoding, shape, size, porosity, conductivity, permittivity, permeability, and stiffness.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides an illustrated view of a composition, in accordance with an exemplary embodiment of the present disclosure.

FIG. 2A provides a magnified view of a metallized bead, in accordance with an exemplary embodiment of the present disclosure.

FIG. 2B provides a magnified view of a metal particle, in accordance with an exemplary embodiment of the present disclosure.

FIG. 3A provides an illustrated view of a nonmetalized bead, in accordance with exemplary embodiments of the present disclosure.

FIG. 3B provides an illustrated view of a metalized bead, in accordance with exemplary embodiments of the present disclosure.

FIG. 4 provides a flow chart of a method for measuring biomarkers, in accordance with an exemplary embodiment of the present disclosure.

FIG. 5 provides a flow chart of a method for measuring biomarkers, in accordance with an exemplary embodiment of the present disclosure.

FIG. 6 provides a cross-sectional view of a system for detecting biomarkers, in accordance with an exemplary embodiment of the present disclosure.

FIG. 7 provides a top view of an aperture inlet and sensing zone, in accordance with an exemplary embodiment of the present disclosure.

FIG. 8 provides a plot of electrical field strength relative to position, in accordance with an exemplary embodiment of the present disclosure.

FIG. 9A provides a plot of impedance changes versus time, in accordance with an exemplary embodiment of the present disclosure.

FIG. 9B provides a plot of impedance changes versus time, in accordance with an exemplary embodiment of the present disclosure.

FIG. 10 provides a plot of impedance at various frequencies versus concentration of a probe molecule, in accordance with an exemplary embodiment of the present disclosure.

FIG. 11 provides a plot of bead count versus concentration of a probe molecule in accordance with an exemplary embodiment of the present disclosure.

FIG. 12 provides a plot of bead count for various samples, in accordance with an exemplary embodiment of the present disclosure.

FIG. 13 provides a plot of high frequency versus low frequency impedance, in accordance with an exemplary embodiment of the present disclosure.

FIG. 14 provides plots of impedance magnitude spectra at various frequencies.

FIG. 15 provides plots of impedance magnitude, impedance phase, impedance magnitude spectra, and impedance phase spectra for various bead radii and various frequencies.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

It must be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms a, an, and the do not denote a limitation of quantity, but rather denote the presence of at least one of the referenced item.

As used herein, the term “and/or” may mean “and,” it may mean “or,” it may mean exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

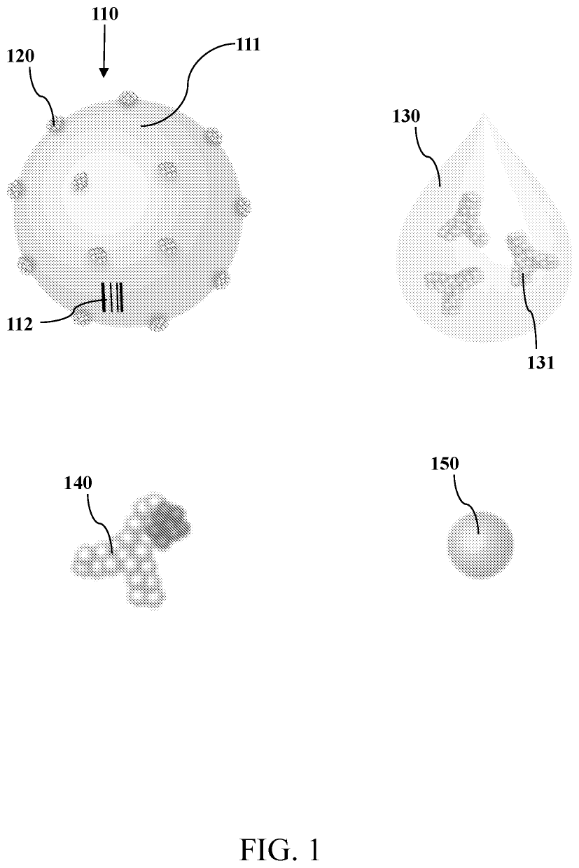

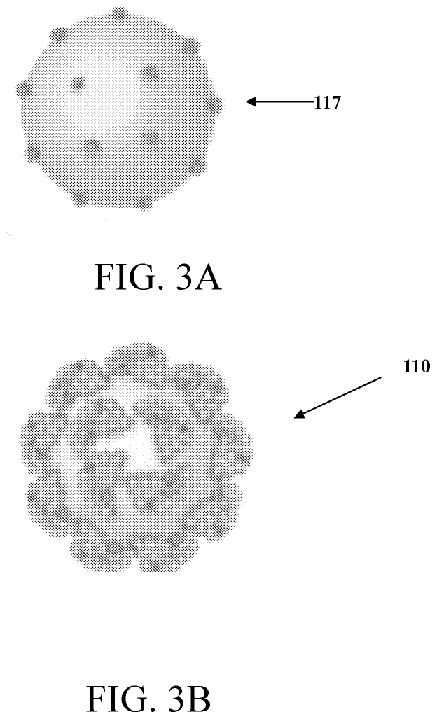

As shown in FIG. 1, FIG. 3A, and FIG. 3B, an exemplary embodiment of the present disclosure provides a composition (100) comprising: a bead (100) having on its surface (111) a molecule of interest (120) configured to bind with a target molecule (131) in a biological sample (130), a probe molecule (140) configured to bind with the target molecule (131), and a metal particle (150) configured to bind with the probe molecule (140).

In any of the embodiments disclosed herein, the bead (110) can comprise an identifying characteristic (112). The identifying characteristic (112) can be one or more of: an electrical impedance signature, optical barcoding, shape, size, porosity, conductivity, permittivity, permeability, and stiffness.

In any of the embodiments disclosed herein, the metal particle (150) can be configured to modify an electrical property of the bead (110).

In any of the embodiments disclosed herein, the bead (110) can comprise one or more of: carboxylated polystyrene, aminated polystyrene, glass, silica, polymethyl methacrylate, and hydrogels.

In any of the embodiments disclosed herein, the bead (110) can be functionalized using EDC-NHS (N-ethyl-N-(3-(dimethylamino) propyl) carbodiimide/N-hydroxysuccinimide) chemistry.

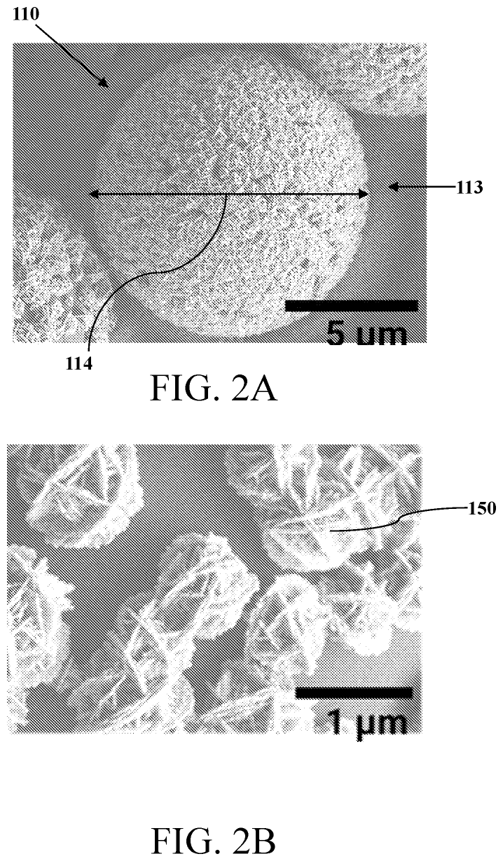

As shown in FIG. 2A, the bead (110) can have a spherical shape (113). The bead (110) can have a diameter (114) of approximately 3 μm.

In any of the embodiments disclosed herein, the bead (110) can have a diameter (114) of approximately 5 μm. In any of the embodiments disclosed herein, the bead (110) can have a diameter (114) of approximately 10 μm. In any of the embodiments disclosed herein, the bead (110) can have a diameter (114) of approximately 20 μm. In any of the embodiments disclosed herein, the bead (110) can have a diameter (114) of from about 3 μm to about 20 μm.

In any of the embodiments disclosed herein, the molecule of interest (120) can comprise one or more of: an antigen, an epitope, an antibody, protein, DNA, RNA, virus, bacteria, and mammalian cell.

In any of the embodiments disclosed herein, the target molecule (131) can comprise one or more of: an antibody, a paratope, a protein, DNA, RNA, a virus, a bacterium, and a mammalian cell.

In any of the embodiments disclosed herein, the biological sample (130) can comprise one or more of: serum, whole blood, saliva, urine, and cerebrospinal fluid.

In any of the embodiments disclosed herein, the composition (100) can further comprise a buffer. The buffer can be a phosphate buffer having a pH of from about 6.0 to about 7.0. The buffer can be a phosphate buffer comprising a pH of approximately 6.4.

In any of the embodiments disclosed herein, the probe molecule (140) can comprise horseradish peroxidase.

As shown in FIG. 2B, in any of the embodiments disclosed herein the metal particle (150) can comprise silver. In any of the embodiments disclosed herein, the metal particle (150) can be a spherical silver nanoparticle of approximately 1 μm in diameter.

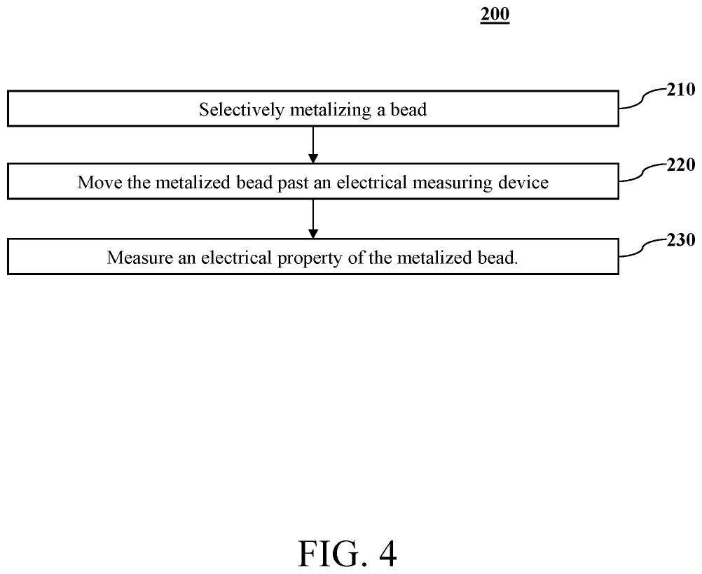

As shown in FIG. 4, an exemplary embodiment of the present disclosure provides a method (200) for measuring biomarkers, the method comprising selectively metalizing (210) a bead (110), moving the metalized bead (110) past an electrical measuring device (314), and measuring (230) an electrical property of the metalized bead (110).

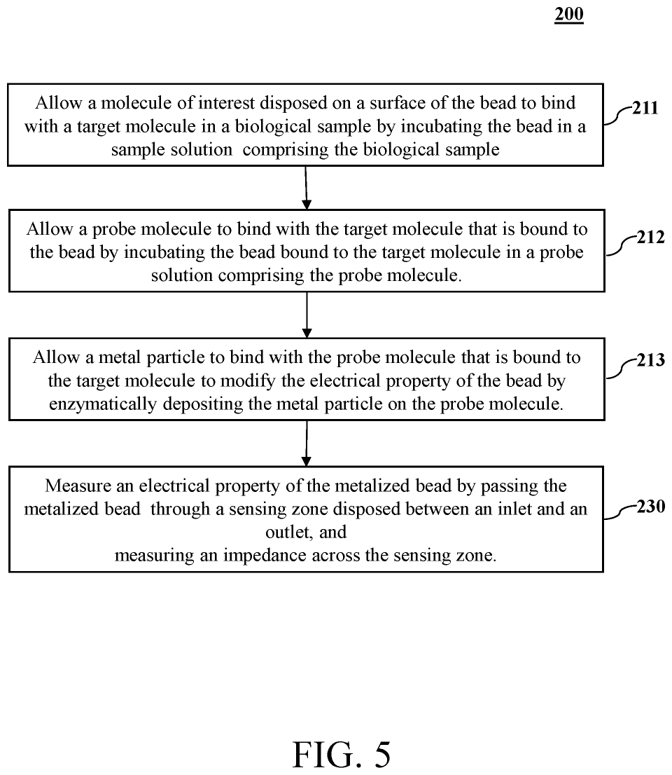

As shown in FIG. 5, in any of the embodiments disclosed herein, selectively metalizing (210) the bead (110) can comprise allowing (211) a molecule of interest (120) disposed on a surface (111) of the bead (110) to bind with a target molecule (131) in a biological sample (130), allowing a probe molecule (140) to bind with the target molecule (131) that is bound to the bead (110), and allowing a metal particle (150) to bind with the probe molecule (140) that is bound to the target molecule (131) to modify the electrical property of the bead (110).

In any of the embodiments disclosed herein, allowing (211) the molecule of interest (120) to bind with the target molecule (131) can comprise incubating the bead (110) in a sample solution (132) comprising the biological sample (130).

In any of the embodiments disclosed herein, incubating (214) the bead (110) in the sample solution (132) can last for approximately 1800 seconds at approximately room temperature.

In any of the embodiments disclosed herein, allowing (212) the probe molecule (140) to bind with the target molecule (131) can comprise incubating (215) the bead (110) bound to the target molecule (131) in a probe solution (141) comprising the probe molecule (140).

In any of the embodiments disclosed herein, incubating (215) the bead (110) bound to the target molecule (131) in the probe solution (141) can last for 3600 seconds at approximately room temperature.

In any of the embodiments disclosed herein, allowing (213) the metal particle (150) to bind with the probe molecule (140) that is bound to the target molecule (131) can comprise enzymatically depositing the metal particle (150) on the probe molecule (140).

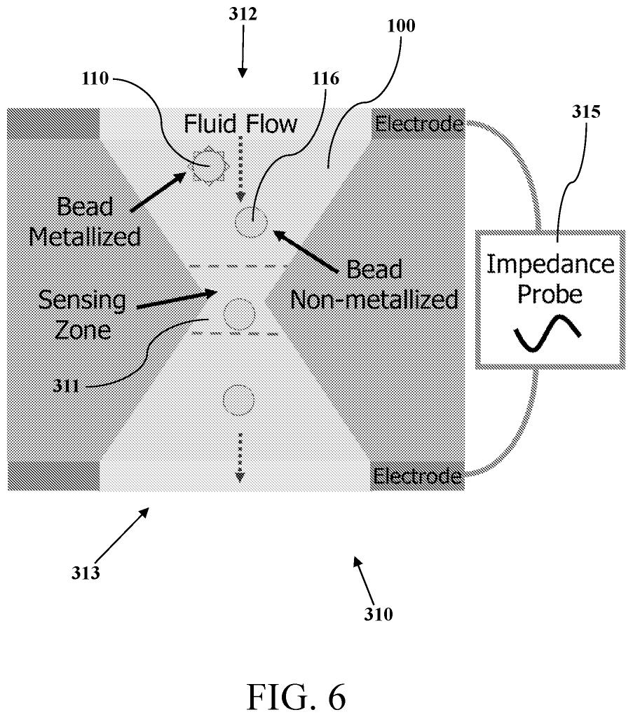

As shown in FIG. 6, in any of the embodiments disclosed herein, measuring (230) an electrical property of the metalized bead (150) can comprise passing (110) the metalized bead (150) through a sensing zone (311) disposed between an inlet (312) and an outlet (313); and measuring an impedance across the sensing zone (311).

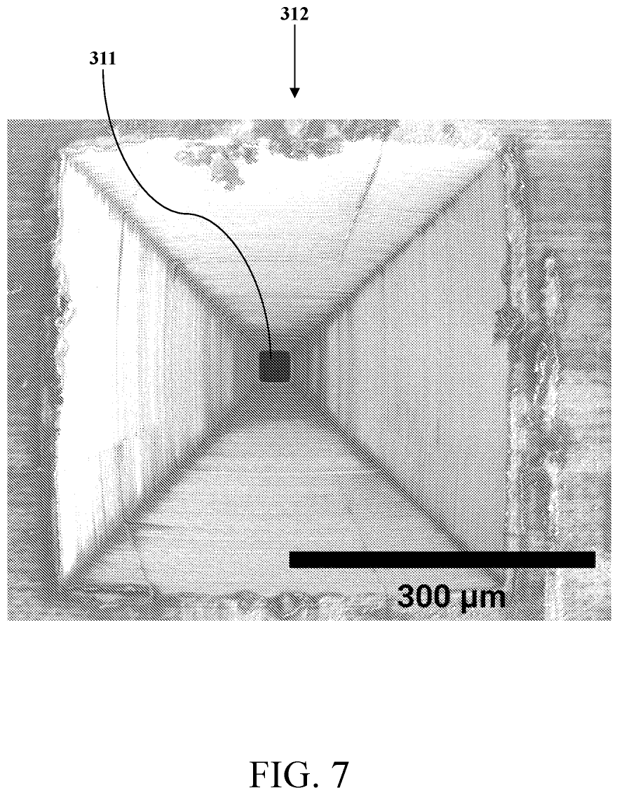

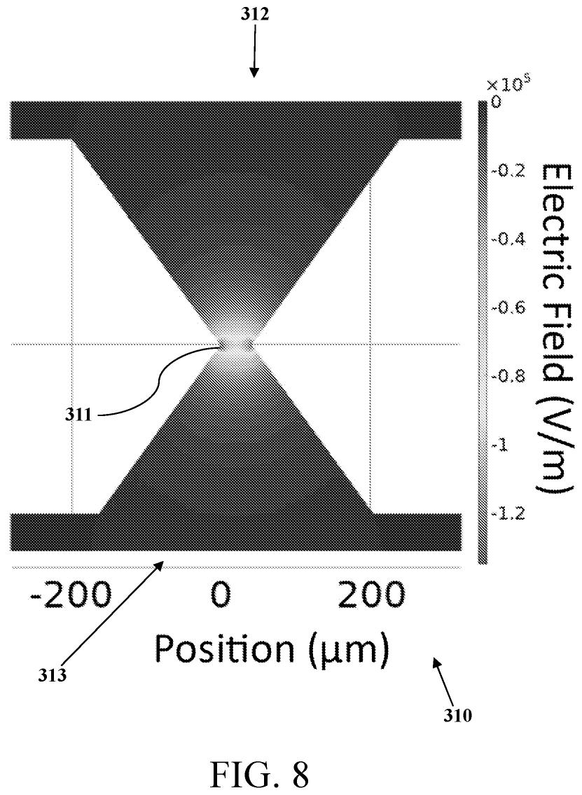

As shown in FIGS. 7 and 8, in any of the embodiments disclosed herein, the inlet (312) can have a pyramidal shape. The sensing zone (311) can have a square shape. The outlet (313) can have a pyramidal shape.

In any of the embodiments disclosed herein, passing the bead (110) through the sensing zone (311) can comprise flowing a bead suspension (115) through the sensing zone (311), the metalized bead (110) suspended in the bead suspension (115).

In any of the embodiments disclosed herein, the bead suspension (115) can be flowed at a rate of approximately 12 microliters per minute.

In any of the embodiments disclosed herein, measuring the impedance across the sensing zone (311) can be performed with a lock-in amplifier.

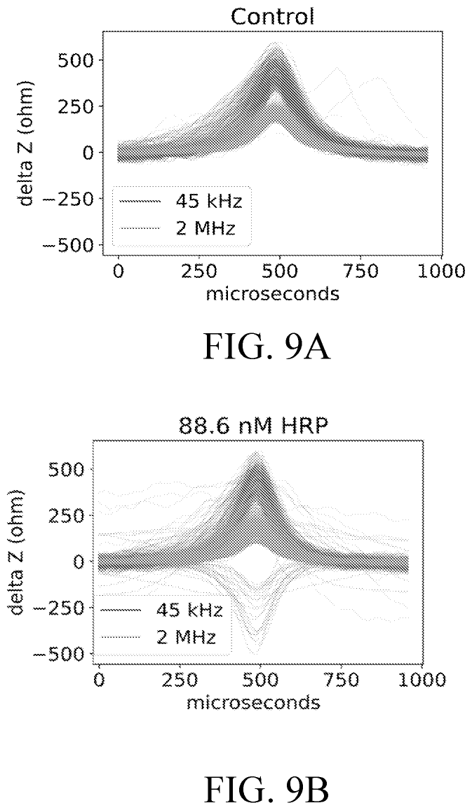

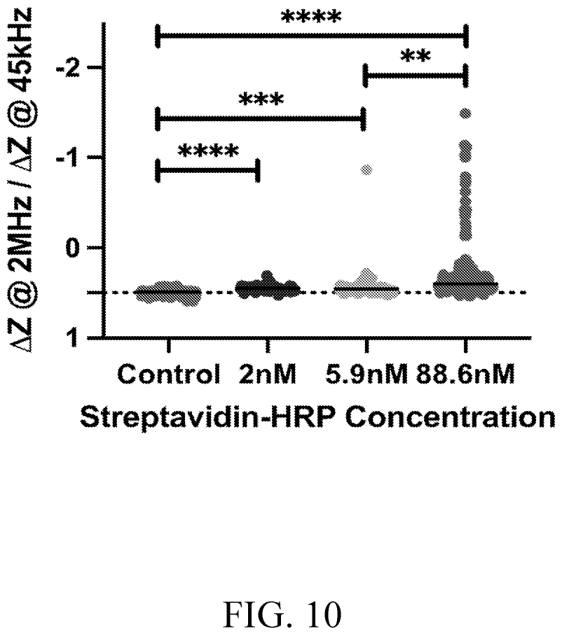

As shown in FIG. 9A, FIG. 9B, and FIG. 10, in any of the embodiments disclosed herein, measuring the impedance can comprise sampling at approximately 100,000 samples per second at one or more frequencies. The one or more frequencies can be between approximately 45 kilohertz and 35 megahertz.

In any of the embodiments disclosed herein, the method can further comprise comparing the electrical property of the selectively metalized bead (110) to an electrical property of a non-metalized bead (116).

In any of the embodiments disclosed herein, the method can comprise identifying a characteristic of the bead (110). The characteristic of the bead (110) can be one or more of: an electrical impedance signature, optical barcoding, shape, size, porosity, conductivity, permittivity, permeability, and stiffness.

In any of the embodiments disclosed herein, the method can further comprise functionalizing the bead using EDC-NHS (N-ethyl-N-(3-(dimethylamino) propyl) carbodiimide/N-hydroxy succinimide) chemistry.

As shown in FIG. 6, an exemplary embodiment of the present disclosure provides a system (300) for detecting biomarkers. The system can comprise a composition (100), an aperture (310), and an electrical property sensor (315). The composition (100) can comprise a bead (110) having on its surface (111) a molecule of interest (120) configured to bind with a target molecule (131) in a biological sample (130). a probe molecule (140) configured to bind with the target molecule (131), and a metal particle (150) configured to bind with the probe molecule (140). The aperture can comprise an inlet (312), an outlet (313); and a sensing zone (311) disposed between the inlet (312) and the outlet (313), wherein the aperture (310) is configured to pass the composition (100) from the inlet (312), through the sensing zone (311), and to the outlet (313). The electrical property sensor can be disposed proximate the sensing zone (311) and configured to sense an electrical property of the bead (110).

In any of the embodiments disclosed herein, the inlet (312) can have a pyramidal shape. The outlet (313) can have a pyramidal shape. The sensing zone (311) can have a square shape.

In any of the embodiments disclosed herein, the sensing zone (311) can be approximately 30 μm by 30 μm.

In any of the embodiments disclosed herein, the aperture (310) can be configured to flow the composition (100) at a rate of approximately 12 microliters per minute.

In any of the embodiments disclosed herein, the electrical property sensor (315) can comprise a lock-in amplifier.

In any of the embodiments disclosed herein, the electrical property sensor (315) further can comprise a processor (316) configured to compare the electrical property of the bead (110) to an electrical property of a non-metalized bead (116). The electrical property sensor (315) can be configured to identify a characteristic of the bead (110), and the characteristic of the bead (110) can be one or more of: an electrical impedance signature, optical barcoding, shape, size, porosity, conductivity, permittivity, permeability, and stiffness.

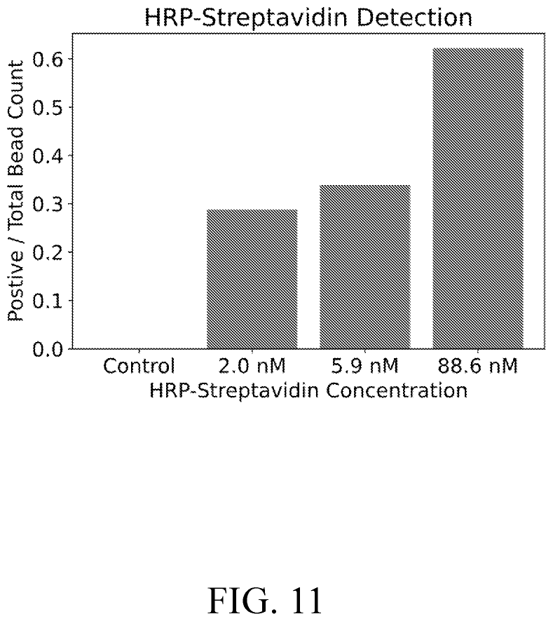

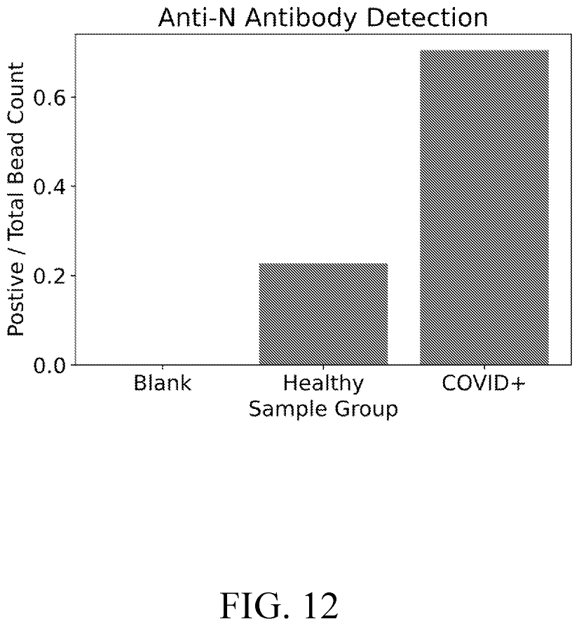

As summarized by the results in FIG. 11-13, multiple beads, some selectively metalized according to the methods herein and some not metalized, can be flowed past the electrical property sensor and counted to determine concentrations of one or more biomarkers. The presence or absence of one or more biomarkers can be determined based on measurements of the electrical property taken by the electrical property sensor.

The following examples further illustrate aspects of the present disclosure. However, they are in no way a limitation of the teachings or disclosure of the present disclosure as set forth herein.

### EXAMPLES

Below, exemplary compositions, methods, and systems for detecting and measuring biomarkers are disclosed. Disclosed herein is an inexpensive, electronic alternative to fluorophores and optics for analyte detection by using a bead-based assay chemistry that mirrors an enzyme-linked immunosorbent assay (ELISA). However, rather than the enzyme-labeled secondary probe catalyzing a color change in solution, a metal deposition onto a bead surface changes the electrical properties of the beads. This enables the use of a simple electrical detection system, based on the ‘Coulter Principle’, to measure the impedance of beads in continuous flow. Here, an in-flow multi-frequency impedance characterization system is developed, using a 3D-printed plastic micro-aperture, to measure microparticles whose impedance characteristics are linked to an immunoassay via the enzymatic metallization. Due to metal deposition and impedance characteristics of metallized beads, they possess a unique impedance spectral signature that enables distinguishing them from non-metallized beads. This method and system can be used in a clinical immunoassay by measuring antibodies directed against the nucleocapsid protein of SARS-COV-2 in convalescent COVID-19 patient serum. A machine learning method can be applied to the measured bead impedance spectra to enables defines a single metric which clearly distinguishes COVID+ and healthy samples. Thus, a fully electronic bead-based immunoassay is realized, which provides a sensitive readout of analyte binding without any intermediate optics.

Magnetic and non-magnetic polystyrene beads functionalized with carboxyl groups are obtained from Spherotech (3.9, 5.6, and 8.2 μm) and Polysciences (4.7, 5.7, 10.3, and 20.1 μm) respectively. To help normalize the surface chemistry stoichiometry between different bead sizes and stock concentrations, the microparticle surface area per solution volume is calculated for each stock solution assuming spherically shaped beads. 25 cm² of beads by surface area are taken from stock and suspended in 1 mL of 1:10 dilution of phosphate buffer, pH 6.2-6.5. 20 μL of ethylene dichloride (EDC) and 20 μL of N-Hydroxysuccinimide (NHS) at 50 mg/mL are added and this solution is incubated for 20 minutes on a rotator. After incubation, one wash cycle is performed with a 1:10 dilution 2-(N-morpholino) ethanesulfonic acid (MES) buffer. Next, 900 μL of 1:10 dilution MES buffer is added with 100 μL of the desired antigen at 0.5 mg/mL and incubated for 2 hours on a rotator. Following this, 2 washes are performed in 1% phosphate-buffered saline (PBS) with 0.1% Tween 20 and 0.1% bovine serum albumin and the beads are stored in this same solution.

Recombinant protein A/G (ThermoFisher Scientific) is immobilized as the antigen. This is incubated for 60 minutes with 2 μL of 1 mg/mL mouse anti-human immunoglobulin (IgG) Fc-Horse Radish Peroxidase (HRP) (SouthernBiotech) in 200 μL of volume. Beads are washed 3 times in de-ionized water before silver deposition is performed with EnzMet (Nanoprobes). 50 μL each of EnzMet solutions A, B, and C are incubated with beads for 4, 4, and 8 minutes respectively.

For clinical sample characterization, SARS-COV-2 Nucleocapsid protein (Sino Biological) is immobilized on the bead surface followed by incubation with 200 μL of 1:100 dilution of pooled COVID-19 patient serum (Innovative Research). This is incubated for 60 minutes with 2 μL of 1 mg/mL mouse anti-human IgG Fc-HRP (SouthernBiotech) in 200 μL of volume. Beads are washed 3 times in de-ionized water before performing silver deposition at the same conditions as above.

The microscale aperture is fabricated by 3-dimensional two-photon-lithography (Nanoscribe). A lock-in amplifier and transimpedance amplifier (HF2LI, HF2TA Zurich Instruments) are used for impedance measurements. Impedance spectra are sampled at 57,000 samples per second at 6 frequencies ranging from 45 kHz to 35 MHz. These time-series signals are passed through a high-pass filter before further analysis. For waveform identification the impedance magnitude at 45 kHz is used in a peak finding algorithm with a threshold of 110 ohms. Timestamps of peaks at this frequency are used to directly index the remaining 5 magnitude and 6 phase time-series measurements. The flow rate of the syringe pump is 12 μL/min. All aggregate measurements reported here are for 300 beads.

Next, finite element analysis (FEA) is performed. To match the baseline of the empirical system lumped elements are added with the electronic circuit interface. Non-metallized beads are simulated as non-conducting spheres and metallized beads are simulated with the electric shielding feature on the non-conducting sphere surface.

A model binding assay is developed to investigate immunobinding and target-probe driven enzymatic metallization on beads and its effect on their electrical properties. Carboxylated polystyrene beads are functionalized, using EDC-NHS chemistry, with recombinant Protein A/G which is used as a model capture antigen for binding enzyme-labeled mouse IgG (HRP-mIgG) as an analyte. This is followed with incubation with the metallization substrate solution. The beads are then washed, dried, and imaged using electron microscopy. A dense nanostructured metal film can be observed on the HRP-mIgG bound beads but not on the control beads. At the nanoscale, the metal layer is found to have the morphology of overlapping dense ‘desert-rose’ like structures. Overall, these results establish that enzyme-labeled probe driven metal deposition on polystyrene beads can be used to create selective and specific immunobinding-driven metallization. HRP-conjugated antibodies bind to immobilized antigens on the bead surface. The HRP enzyme catalyzes metal deposition.

Next, a microfluidic system is provided for high-throughput, sensitive, in-flow impedance characterization of individual beads in suspension. This is performed by passing the bead suspension pass through a microscale aperture placed between two electrodes. The aperture, shaped as an inverted double-pyramid, localizes the electric field to a small region. Thus, the impedance measured between the two electrodes is highly dependent on the electrical properties of this restricted ‘sensing zone’ near the smallest cross-section of the aperture. This allows in-flow measurement of single beads in the sensing zone without crosstalk from other beads outside it.

The fabricated aperture is 30×30 μm at the narrowest part. The overall sensor assembly comprises gold-plated through-holes (0.6 mm diameter) on standard printed circuit boards which are aligned with the aperture and glued together along with fluid connections to a reservoir and connection to a syringe pump that pulls fluid through the aperture. The impedance across the electrodes is measured simultaneously at six frequencies using a lock-in amplifier. When a bead passes through the aperture, the impedance increases transiently, which appears as a symmetric waveform rising as the bead enters the sensing zone, reaching a maximum or peak value and then falling as it exits.

To characterize the operation of the microfluidic impedance measurement system, the frequency response of the system is measured for various levels of salt concentrations in the buffer fluid flowing through the aperture without any beads. The system shows a flat (i.e. resistive) magnitude response up until a salt concentration-dependent cutoff frequency after which it declines indicating that capacitive effects dominate. Additionally, inductive effects appear at the high frequency end. Also, as more salt is added, the impedance is found to decrease as expected due to increasing conductivity of the fluid. A multiphysics impedance model of the system, including the FEA model of the aperture itself, serves to compare fluid conductivity set to that of 1% saline. This model allows the extraction of the lumped parameters representing both the capacitive and inductive parasitic effects and the fitted model output matched the corresponding measured impedance spectrum. This coupled FEA and circuit computed model is used hence as the baseline for modeling bead impedances as well.

When a bead passes through the aperture, the impedance increases transiently which appears as a symmetric waveform rising as the bead enters the sensing zone, reaching a maximum or peak value and then falling as it exits.

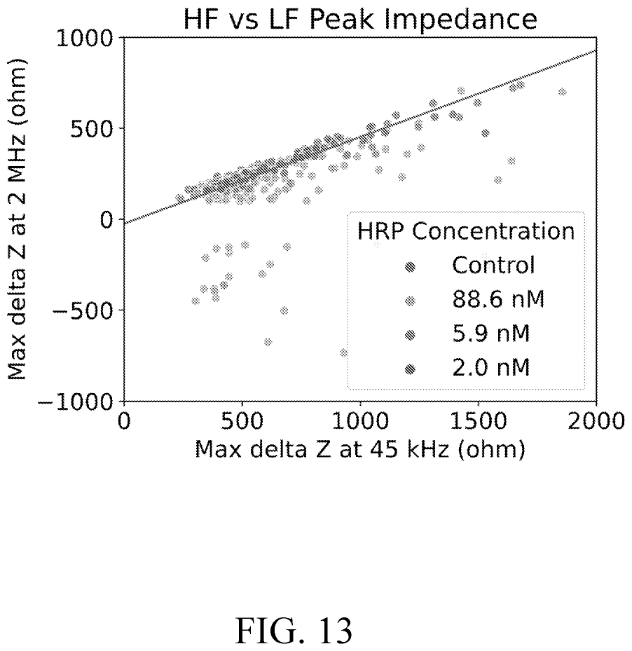

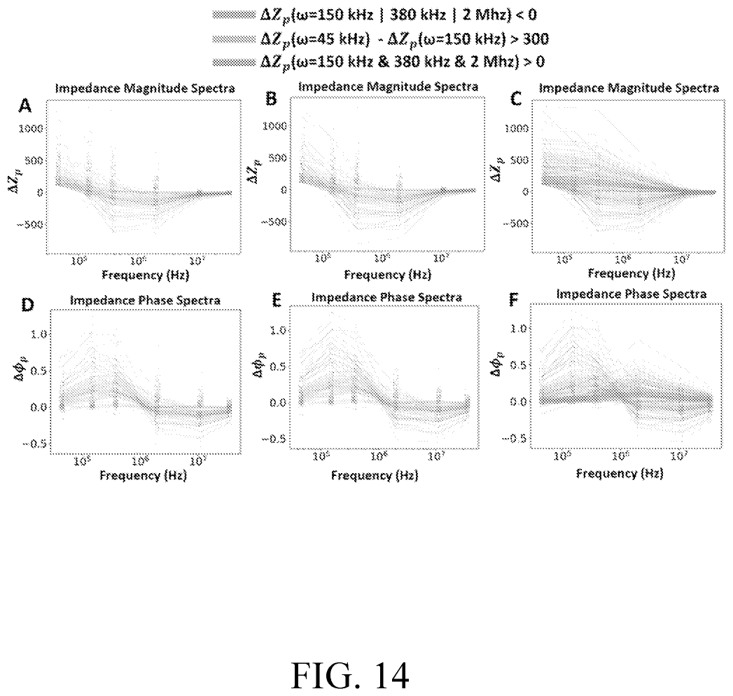

Impedance magnitude and phase waveforms are recorded for seven different sizes of non-metallized polystyrene beads ranging in diameter from 3.9 to 20.1 μm through the aperture. Peak change in magnitude (ΔZₚ) and phase (Δϕₚ) values are obtained for each. FIG. 13 shows the variation of ΔZₚ and Δϕₚ with bead radius at two selected frequencies (45 kHz & 2 MHz). As expected, ΔZₚ rises with bead size as larger non-conductive beads displace more of the conductive fluid filling the sensing zone. Δϕₚ remains low showing that the impedance change due to beads is mostly resistive at these two frequencies with higher bead sizes showing slightly higher capacitive phase change. These results are compared with multiphysics simulation results, for non-conducting spheres of different sizes passing through the aperture. ΔZₚ is flat at lower frequencies but then decreases at higher frequencies. Δϕₚ increases and peaks ˜2 MHz and then declines. Based on these observed spectra, a simplified lumped element circuit model for the non-metallized beads which is found to agree well with the measurements. A capacitive effect of the bead is also implied by the impedance phase change peaking at 2 MHz and is assumed to be due to the non-conducting bead acting as an additional dielectric between the relatively conductive fluid above and below it. This bead capacitance is modeled as parallel to the baseline capacitance.

Metallized beads (8.2 μm) have significantly different impedance and phase spectra compared to non-metallized beads. ΔZₚ for metallized beads is, overall, significantly lower than non-metallized beads and even becomes negative at some frequencies. This implies that the metallized beads are more conductive than the surrounding fluid they replace. However, strikingly, this negative dip is found to be a frequency-specific effect and does not occur across the spectrum. This results in a unique shape of the ΔZₚ spectra as well for the metallized beads, which is distinguishable from that of the non-metallized beads. Δϕₚ of metallized beads has a positive peak at a lower frequency than the 2 MHz peak of non-metallized beads and takes on more negative values at 2 MHz. This shows that metallized and non-metallized beads can be distinguished based on their unique impedance spectra.

Next, a lumped element circuit model, to match the observed experimental results, is developed. For this, the metallized bead surface is considered as a resistor in series with a capacitor, in parallel to the non-conductive bead circuit model. This model is found to match the general shape of the impedance and phase spectrum of the metallized beads including the frequency-specific negative impedance signature. The many individual metal spheroids of ‘desert-rose’ like shape create an intricate mesh of metal and fluid and that an equivalent capacitance may be expected from the metal-electrolyte interfaces and the fluid between conducting metal spheroids instead of a simple conducting layer as modeled in computer simulations. Thus, the distinctive nanostructure of the enzymatic metallization layer may contribute to the unique impedance signature of the metallized beads.

Additionally, scanning electron microscope (SEM) images of metallized beads showed that there can be significant variation in the degree of metallization of bead surface. The impedance spectra are used to identify some possible criteria to segregate sub-populations within the metallized beads. A distinct subset of beads which show a sharp drop-off from relatively large positive ΔZₚ at the lowest frequency to produce relatively large negative ΔZₚ at intermediate frequencies. Notably, the Δϕₚ for this subset group are correspondingly extreme as well producing maxima at lower frequencies followed by a sharper drop-off compared to other beads within the negative ΔZₚ set. These frequency-specific sharp drop-offs and flip in sign of ΔZₚ indicate that these metallized beads did not simply undergo a change in overall bead conductivity but demonstrate the unique effect of the nanostructured enzymatic metallization film on the bead surface. This subset of beads are the ones that show the densest metallization under the SEM and term them the Mₕᵢ subset. The rest of beads in the negative ΔZₚ set are terms termed M_{mcd} and are likely the ones with relatively less dense metallization. Overall, the identification of these bead subsets with distinct impedance signatures within the metallized bead impedance spectra indicate the ability of this high-throughput, single bead impedance spectrometry technique in identifying heterogeneity in the metallization and classifying beads based on it and potentially quantifying such effects as well.

Finally, the bead-based immunobinding, enzymatic metallization, and the microfluidic bead impedance spectrometry techniques to generate unique impedance signatures of individual metallized beads is integrated into a fully electronic bead-based immunoassay to measure SARS-COV-2 antigen-specific antibodies from convalescent COVID-19 patient serum. Among the different beads characterized earlier, the 8.2 μm magnetic beads are used for this assay based on a trade-off between higher signal-to-noise ratio offered by larger bead sizes versus increased risk of clogging of the aperture observed with even larger beads as well as the ease of handling of magnetic beads. Beads are functionalized with the SARS-COV-2 Nucleocapsid (N) protein, incubated with COVID positive (COVID+) serum or pre-pandemic healthy serum as a negative control. They are then probed with mIgG-HRP directed against human IgG (hIgG) in order to measure anti-N hIgG levels in the sera. Finally, they are incubated with the metallization substrate. Beads incubated with COVID+ serum show high levels of metallization while those incubated with healthy serum show significantly lower metallization.

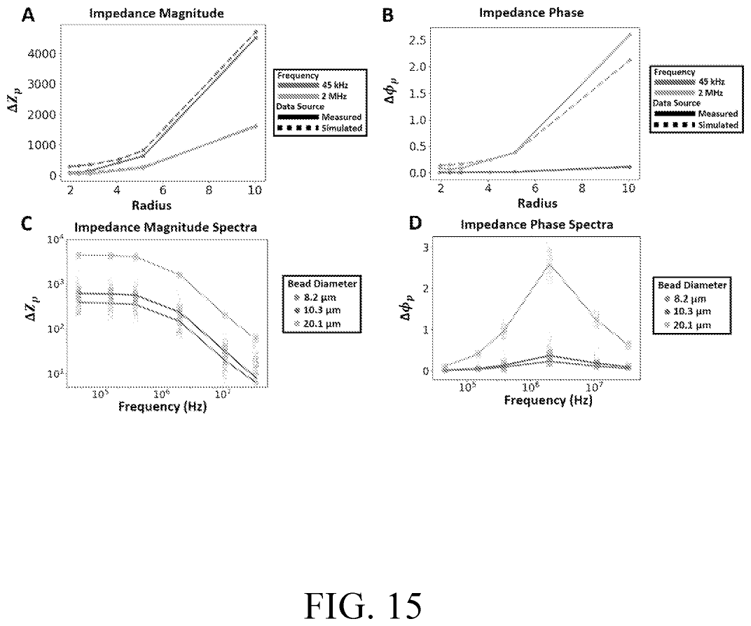

Both bead sets are then characterized using the microfluidic impedance spectrometry system. ΔZₚ and Δϕₚ spectra for both are shown in FIG. 15. A machine-learning method is used to find a singular diagnostic metric from these spectra, to distinguish COVID+ versus healthy samples.

The bead-based immunoassay disclosed herein links analyte binding to enzymatic metallization on bead surfaces to produce ‘impedance-labelled’ beads in contrast to widely used fluorescently labeled beads. A nanostructured ‘desert rose’ morphology is observed for the enzymatic metallization layer on the beads. In-flow bead impedance sensing is performed using a 3D-printed microscale aperture to capture the impedance spectrum for each individual beads in a high-throughput manner. Metallized beads are found to have a distinctive impedance spectral signature with frequency-specific negative dips in impedance change which are not found for non-metallized beads. Thus, metallized, and non-metallized beads can be clearly distinguished using this electronic signature. Additionally, distinct subsets of metallized beads with high, medium and low metallization-based impedance signatures are found.

A finite element model for the bead impedance measurement scheme accurately predicted non-metallized bead s impedances but not for the metallized bead impedances shows that their impedance spectra cannot be modeled via non-conductive beads with a purely conductive metallization layer. Instead, a lumped element circuit model which models the metallization layer as a combined resistive and capacitive element matches the measured impedance spectra better for the metallized beads.

In summary, this example bead-based immunoassay provides a sensitive direct electronic readout without the use of any intermediate optics. Analyte binding to antigen-coated microparticles is converted to probe-directed enzymatically amplified silver metallization on microparticle surfaces. The microparticles are then characterized in a high-throughput manner via electrical impedance spectra captured as they flow through a 3-D printed plastic micro-aperture. Metallized microparticles are found to have unique impedance signatures. This enables an electronic readout of the silver metallization density on microparticle surfaces and hence analyte concentration as well. This scheme is used to measure the antibody response to the viral nucleocapsid protein in convalescent COVID-19 patient serum. Beyond this current demonstration. this technology can be applied to the sensing of any biomarker that can be specifically bound to with a probe, such as proteins, antigens, cells, DNA, RNA, viruses, and bacterium. In regards to immunoassays, this technology can also be multiplexed by using ‘impedance-barcoded’ beads, where an identifiable property of the bead can be altered that is independent of the electronic signal produced by analyte binding. For example, this could be the shape, porosity, size, conductivity or permittivity or a combination thereof of the beads.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A composition comprising:
a bead having on its surface a molecule of interest configured to bind with a target molecule in a biological sample;
a probe molecule configured to bind with the target molecule; and
a metal particle configured to bind with the probe molecule.

2. The composition of claim 1, wherein the bead has:
an identifying characteristic; and
a spherical shape with a diameter of from about 3 μm to about 20 μm;
wherein the identifying characteristic is selected from a group consisting of an electrical impedance signature, optical barcoding, shape, size, porosity, conductivity, permittivity, permeability, and stiffness.

3. (canceled)

4. The composition of claim 2, wherein the metal particle is configured to modify an electrical property of the bead.

5. The composition of claim 4, wherein the metal particle comprises at least one of a silver nanoparticle, a gold nanoparticle, or an iron nanoparticle

6. The composition of claim 2, wherein the bead comprises at least one of carboxylated polystyrene, aminated polystyrene, glass, silica, polymethyl methacrylate, or hydrogels.

7. The composition of claim 6, wherein the bead is functionalized using EDC-NHS (N-ethyl-N′-(3-(dimethylamino) propyl) carbodiimide/N-hydroxy succinimide) chemistry.

8.-13. (canceled)

14. The composition of claim [[1]] 2, wherein the molecule of interest comprises at least one of an antigen, an epitope, an antibody, protein, DNA, RNA, virus, bacteria, or mammalian cell.

15. The composition of claim 2, wherein the target molecule comprises at least one of an antibody, a paratope, a protein, DNA, RNA, a virus, a bacterium, or a mammalian cell.

16. The composition of claim 2, wherein the biological sample comprises at least one of serum, whole blood, saliva, urine, or cerebrospinal fluid.

17.-19. (canceled)

20. The composition of claim 2 further comprising:
a phosphate buffer having a pH of from about 6.0 to about 7;
wherein;
the bead comprises at least one of carboxylated polystyrene, aminated polystyrene, glass, silica, polymethyl methacrylate, or hydrogels;
the molecule of interest comprises at least one of an antigen, an epitope, an antibody, protein, DNA, RNA, virus, bacteria, or mammalian cell;
the target molecule comprises at least one of an antibody, a paratope, a protein, DNA, RNA, a virus, a bacterium, or a mammalian cell;
the biological sample comprises at least one of serum, whole blood, saliva, urine, or cerebrospinal fluid;
the probe molecule at least one of horseradish peroxidase or alkaline phosphatase; and
the metal particle is configured to modify an electrical property of the bead and comprises at least one of a silver nanoparticle, a gold nanoparticle, or an iron nanoparticle.

21. A method comprising:
moving the composition of claim 1 past an electrical measuring device; and
measuring an electrical property of the composition.

22. The method of claim 21 further comprising:
composing the composition by:
disposing the molecule of interest on the surface of the bead binding the molecule of interest to the target molecule;
binding the probe molecule to the target molecule;
binding the metal particle to the probe molecule; and
modifying the electrical property of the bead.

23. The method of claim 22, wherein: p1 binding the molecule of interest to the target molecule comprises incubating the bead in a sample solution comprising the biological sample;
binding the probe molecule to the target molecule comprises incubating the bead bound to the target molecule in a probe solution comprising the probe molecule; and
binding the metal particle to the probe molecule that is bound to the target molecule comprises enzymatically depositing the metal particle on the probe molecule.

24. The method of claim 23, wherein at least one of:
incubating the bead in the sample solution lasts for approximately 1800 seconds at approximately room temperature; or
incubating the bead bound to the target molecule in the probe solution lasts for approximately 3600 seconds at approximately room temperature.

25.-28. (canceled)

29. The method of claim 21, wherein measuring the electrical property of the composition comprises:
passing the composition through a sensing zone, the sensing zone disposed between an inlet and an outlet; and
measuring an impedance across the sensing zone.

30. The method of claim 29, wherein at least one of:
the inlet has a pyramidal shape;
the outlet has a pyramidal shape; or the sensing zone has a square shape.

31. The method of claim 29, wherein passing the composition through the sensing zone comprises flowing a bead suspension through the sensing zone; and
wherein the composition is suspended in the bead suspension.

32. The method of claim 31, wherein the bead suspension is flowed at a rate of approximately 12 microliters per minute.

33. The method of claim 21, wherein at least one of:
measuring the electrical property of the composition comprises passing the composition through a sensing zone and measuring an impedance across the sensing zone with a lock-in amplifier;
measuring the electrical property of the composition comprises passing the composition through the sensing zone and measuring the impedance across the sensing zone with the lock-in amplifier sampling at approximately 100,000 samples per second at one or more frequencies; or
measuring the electrical property of the composition comprises passing the composition through the sensing zone and measuring the impedance across the sensing zone with the lock-in amplifier sampling at approximately 100,000 samples per second at frequencies between approximately 45 kilohertz and 35 megahertz.

34.-35. (canceled)

36. The method of claim 21 further comprising at least one of:
comparing the electrical property of the composition to an electrical property of a non-metalized bead;
identifying a characteristic of the composition, wherein the characteristic is selected from a group consisting of an electrical impedance signature, optical barcoding, shape, size, porosity, conductivity, permittivity, permeability, and stiffness; or
functionalizing the composition using EDC-NHS (N-ethyl-N′-(3-(dimethylamino) propyl)carbodiimide/N-hydroxy succinimide) chemistry.

37.-38. (canceled)

39. A system comprising:
the composition claim 1;
an aperture comprising:
an inlet;
an outlet; and
a sensing zone disposed between the inlet and the outlet; and
an electrical property sensor disposed proximate the sensing zone and configured to sense an electrical property of the composition;
wherein the aperture is configured to pass the composition from the inlet, through the sensing zone, and to the outlet.

40.-65. (canceled)
